(19) United States
(12) Patent Application Publication
(10) Pub. No.: US 2019/0307661 A1
(43) Pub. Date: Oct. 10, 2019

(54) Antiperspirant Chalk Composition and Method

(71) Applicant: Yousif Saroya, Astoria, NY (US)

(72) Inventor: Yousif Saroya, Astoria, NY (US)

(21) Appl. No.: 16/372,665

(22) Filed: Apr. 2, 2019

Related U.S. Application Data
(60) Provisional application No. 62/652,799, filed on Apr. 4, 2018.

(51) Int. Cl. A61K 8/28; A61K 8/26; A61Q 15/00

(52) U.S. Cl. CPC A61K 8/28; A61K 8/26; A61K 8/046; A61K 2800/30; A61K 2800/592; A61Q 15/00

(57) ABSTRACT

A composition/formula mixture reduces perspiration and enhances grip for an athlete. The composition consists of a chalk component and an aluminum or zirconium additive, preferably one that is chlorohydrate-based, said additive being 35% or less of the composition. The composition may be applied as a powder, a liquid, a roll on, a solid or a spray. A method for using the aforesaid composition to improve an athlete's gripping performance is also disclosed.

### CROSS REFERENCE TO RELATED APPLICATION

This application is a perfection of pending U.S. Provisional Application Ser. No. 62/652,799, filed on Apr. 4, 2018, the disclosure of which is fully incorporated herein.

### BACKGROUND OF THE INVENTION

### 1. Field of the Invention

This invention relates to an improved composition for athletic use. More particularly, it relates to an improved chalk-based composition that exhibits better perspiration deterrence AND improved gripping for certain sports particularly rock and wall climbing. It is meant for applying to human skin on one or more of: the hands, feet, arms, underarms, groin and other body areas. It may be applied as either a powder, solid stick, roll on or spray.

### 2. Relevant Art

Disclosures pertinent to the present invention include, but are not limited to, the following references: U.S. Pat. No. 4,536,413, U.S. Pat. No. 4,572,690, U.S. Pat. No. 6,656,257, U.S. Pat. No. 7,201,914, US Published Application No. 2004/019728, US Published Application No. 2006/0045860, US Published Application No. 2012/0321579, US Published Application No. 2016/0338917, PCT Application No. WO03005981, European Patent Application No. 1080715 and European Patent Application No. 1175885.

The present invention is directed to a composition (and method of use) for controlling, preventing, ameliorating and/or treating perspiration contamination on the skin of human hands, arms, underarms, feet, groin and other body areas. It will also reduce or control the symptoms and side effects of such perspiration, like reduced gripping for certain athletic activities.

Some undesirable skin maladies are associated with conditions that promote or allow unchecked growth of bacteria on or within perspiring skin. For example, acne occurs when increased production of skin lipids causes the pores of the skin to close or be blocked. This causes further skin secretions (primarily perspiration and lipids) to back up within the pores. Normal flora can then proliferate in such pores, fueled by the body's own secretions of moisture (perspiration) and fuel (lipids). The body responds to this mounting bacterial load and build up of skin secretions with an inflammatory reaction, producing the hallmark acne lesions.

Other skin conditions are also produced or exacerbated by the combination of microbial infection, chronic moisture and the host's response, including tinea pedis (athlete's foot), tinea cruris (jock itch), psoriasis, seborrhea and eczema.

It has been observed that aluminum and zirconium salts, especially those related to aluminum chlorohydrate and zirconium chlorohydrate, can inhibit the production of the perspiration and lipids. When applied to the human skin, these compounds form a colloidal plug or gel that prevents release of perspiration and lipids. Since these fluids are not released, the body does not produce more. This not only keeps the skin dry, but also prevents microbes on the skin from feeding off of the fluids and damaging the skin.

Accordingly, it is an object of the present invention to overcome prior perspiration prevention failures and to provide a new composition (and related method of use thereof) that will control, prevent, ameliorate, or otherwise treat undesirable topical (i.e. outer skin) perspiration on the hands, arms, underarms, feet, groin, and other body areas of its users.

### SUMMARY OF THE INVENTION

The present invention is directed to a composition (and method of use) for an improved antiperspirant agent that will better control, prevent, ameliorate, or otherwise treat undesirable perspiration of the hands, arms, feet, groin and other areas of the human body. It will also reduce or control symptoms and side effects of such perspiration like reduced gripping for certain athletic (and/or work) activities.

One embodiment of the present invention comprises combining a known topical dessicant, i.e., chalk, with an additive (preferably aluminum chlorohydrate) such that the effects of such combination are superior to the use of either active component alone.

### DETAILED DESCRIPTION OF PREFERRED EMBODIMENTS

The present invention comprises a composition and method for controlling, preventing, ameliorating, or otherwise treating undesirable topical perspiration on the skin of human hands and feet. It may also be used on other body areas including the arms, underarms, feet and/or groin of its users to reduce or control the symptoms/side effects of such perspiration. A key feature of the present invention is a combination of a known topical antiperspirant and a special additive (in preferred quantities), wherein the effects of such combination are superior to the use of either active component alone. The term antiperspirant is defined herein as an agent that checks, reduces or eliminates perspiration.

Rock climbing is an outdoor and indoor sport in which athletes ascend natural rock cliffs, faces, boulders as well as artificial gym climbing holds with the goal of not falling off the rock face. In order to ascend, climbers rely on their grip/ frictional contact to tactfully move from hold to hold. Perspiration slickens the skin on the hands thus reducing friction and grip ability.

Climbers routinely use chalk (magnesium carbonate, calcium carbonate, and other chalk compounds) to absorb perspiration to enhance grip/ frictional contact on rocks and climbing holds. At times of increased perspiration (due to any amount of circumstances e.g. increased environmental temperature, humidity, mental stress, etc.) a climber must constantly reapply chalk to maintain optimal grip/friction. The act of applying chalk during the midst of climbing is taxing on the body (one must let go of the rock face with one hand to reapply, while the other hand does twice the work to continue to hold onto the rock formation). This ultimately leads to either a more taxing/strenuous ascent or complete failure to ascend due to wasted energy spent on chalk reapplication instead of the act of climbing. This invention introduces an antiperspirant agent (a type of aluminum or zirconium salt) to limit/suppress perspiration. This in turn limits the need for chalk reapplication, thus improving product efficacy and ultimately increasing climbing ability.

### Preferred Formulation

A base component of magnesium carbonate with an addition of about 5-35% of aluminum chlorohydrate, preferably about 10-30% and most preferably about 15-25%. An ideal mixture would be an 80-20 combination of the two main components.

Other chalk compounds such as calcium carbonate may be substituted or added as well as other aluminum- and zirconium-based antiperspirant compounds.

Substitute ingredients for aluminum chlorohydrate include: aluminum chloride, aluminum chloride hexahydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium glycine, aluminum zirconium pentachlorohydrex gly, aluminum zirconium tetrachlorohydrate, aluminum zirconium tetrachlorohydrex gly, aluminum zirconium trichlorohydrate and/or aluminum zirconium trichlorohydrex glycine.

Why certain ingredients and ranges were excluded:

A range of 5-35% was chosen due to these reasons: (1) Test subjects could not notice the effects of the antiperspirant in formulations that contained less than 5% antiperspirant. (2) At or above 5% subjects experienced reduced perspiration. (3) At above 35%, the texture of the formulation started to exhibit a grittier texture that reduced friction and grip ability.

Rosin was tested as an additional grip enhancer, but was ultimately not included in the formulation due to the fact that this ingredient left a sticky/tacky residue on climbing holds and the skin itself.

Silica powder was considered as an additional perspiration absorber, but further research showed that if inhaled, silica powder can cause Silicosis (scarring of lung tissue), increased risk of TB, lung cancer, and other forms of lung disease. See, https.//www.osha.gov/dsg/topics/silicacrystalline/

Talc and corn starch were also tested as additional perspiration absorbers. These ingredients, however, reduced friction and gripping ability due to their slick/slippery texture.

### Example 1

### Control of Perspiration on Un-Gloved Human Hands

In this example, hands were treated with an antiperspirant powder containing about 20% aluminum chlorohydrate, and the balance magnesium carbonate. Once applied, participants noticed improved hand grip and reduced perspiration in climbing applications and when performing weightlifting exercises, i.e., reduced slippage on a pull up bar and a sturdier grip on barbells/dumbbells.

### Example 2

### Use of a Lotion-Based Alternative

Another test will be done using a lotion alternative applied to both hands.

### Example 3

### Spray or Aerosol Formula

Yet another test will be performed using an aerated formula that can be applied by spraying onto both hands.

These examples and applications are provided for illustrative purposes, as the present invention is not limited to the recited examples and applications and includes other modalities and formulations known to those skilled in the art.

Any combined antiperspirant composition of the present invention may comprise, for example, a cream, gel, hydrogel, liquid, liquid spray, liquid suspension, lotion, ointment, aerosol spray, powder, aerosol powder, or similar formulation. For use in gloves, shoes, athletic equipment, and similar items, the composition may be applied to the relevant skin areas prior to donning such items, or may be applied to the items themselves (i.e., the gloves, etc.) prior to their donning. Such composition may be added to such items at the time of manufacture (for example, inside protective gloves during manufacture). In some instances (such as on the hands), it is preferable that the composition comprises a liquid that does not contain moisturizing components, so as to avoid loss of dexterity or grip.

Moreover, one or more of the active ingredients may be encapsulated into a time-release vehicle (e.g., liposome or similar vehicle) to enhance its longevity of efficacy.

Having described the presently preferred embodiments, it is to be understood that this invention may otherwise be covered by the scope of the claims that follow.

What is claimed is:

1. An improved composition for perspiration deterrence on human body skin, said composition comprising: (a) a chalk component, combined with (b) about 5-35% of an aluminum- or zirconium-based additive.

2. The composition of claim 1 wherein the aluminum- or zirconium-based additive is selected from the group consisting of: aluminum chloride, aluminum chloride hexahydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium glycine, aluminum zirconium pentachlorohydrex gly, aluminum zirconium tetrachlorohydrate, aluminum zirconium tetrachlorohydrex gly, aluminum zirconium trichlorohydrate, aluminum zirconium trichlorohydrex glycine and combinations thereof.

3. The composition of claim 1 wherein the chalk component is selected from magnesium carbonate, calcium carbonate and combinations thereof.

4. The composition of claim 1, which contains about 10-30% of the aluminum- or zirconium-based additive.

5. The composition of claim 4, which contains about 15-25% of the aluminum- or zirconium-based additive.

6. The composition of claim 1, which is free of: rosin, silica powder, talc and corn starch.

7. The composition of claim 1, which is applied as a powder or a liquid.

8. The composition of claim 1, which is applied as a roll on solid.

9. The composition of claim 1, which is applied as a spray.

10. A topical composition for applying to human skin and improving gripping when performing an athletic activity, said composition comprising: (a) a chalk component, combined with (b) about 5-35% of an aluminum- or zirconium-chlorohydrate additive selected from the group consisting of: aluminum chloride, aluminum chloride hexahydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium glycine, aluminum zirconium pentachlorohydrex gly, aluminum zirconium tetrachlorohydrate, aluminum zirconium tetrachlorohydrex gly, aluminum zirconium trichlorohydrate, aluminum zirconium trichlorohydrex glycine and combinations thereof.

11. The composition of claim 10, which may be applied to a human hand or foot prior to performing the athletic activity.

12. The composition of claim 10 wherein the chalk component is selected from magnesium carbonate, calcium carbonate and combinations thereof.

13. The composition of claim 10, which contains about 10-30% of the aluminum- or or zirconium-chlorohydrate additive.

14. The composition of claim 13, which contains about 15-25% of the aluminum- or zirconium-chlorohydrate additive.

15. The composition of claim 10, which is applied as a powder, a liquid, a roll on solid or a spray.

16. A method for improving an athlete's gripping performance, said method comprising:
(a) providing a composition comprising:
(i) a chalk component selected from magnesium carbonate, calcium carbonate and combinations thereof, said chalk component being combined with
ii) about 5-35% of an aluminum- or a zirconium-based additive selected from the group consisting of: aluminum chloride, aluminum chloride hexahydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium glycine, aluminum zirconium pentachlorohydrex gly, aluminum zirconium tetrachlorohydrate, aluminum zirconium tetrachlorohydrex gly, aluminum zirconium trichlorohydrate, aluminum zirconium trichlorohydrex glycine and combinations thereof; and
(b) applying the composition to the athlete's hands and/or feet.

17. The method of claim 16 wherein the chalk component of the composition contains about 10-30% of the aluminum- or zirconium-based additive.

18. The method of claim 17 wherein the chalk component of the composition contains about 15-25% of the aluminum- or zirconium-based additive.

19. The method of claim 16 wherein the composition is applied as a powder, a liquid, a roll on solid or a spray.
